(19) United States
(12) Patent Application Publication
(10) Pub. No.: US 2022/0331149 A1
(43) Pub. Date: Oct. 20, 2022

(54) MANDIBULAR ADVANCEMENT DEVICES, KITS, SYSTEMS, AND METHODS

(71) Applicant: Peter Balacky, Pacific Palisades, CA (US)

(72) Inventor: Peter Balacky, Pacific Palisades, CA (US)

(21) Appl. No.: 17/659,640

(22) Filed: Apr. 18, 2022

Related U.S. Application Data
(60) Provisional application No. 63/176,821, filed on Apr. 19, 2021.
(60) Provisional application No. 63/232,583, filed on Aug. 12, 2021.

(51) Int. Cl. A61F 5/56

(52) U.S. Cl. CPC A61F 5/566

(57) ABSTRACT

Devices, kits, systems, and methods for forward adjustment of a mandible of an individual for prevention, diagnosis, and treatment of sleep apnea, snoring, and other conditions, diseases, and disorders characterized at least in part by a rearwardly positioned mandible. A mandibular advancement device (MAD) includes an upper guard, a lower guard, and an adjustment piece that engages the upper guard and the lower guard to limit rearward movement of the lower guard relative to the upper guard during use. As the upper guard and the lower guard are worn by the individual, rearward movement of the mandible is also limited. A plurality of different adjustment pieces defines a plurality of different positions of the lower guard relative to the upper guard for gradual and correct titration of the mandible to gradually open and stabilize the oropharyngeal airway.

### CROSS-REFERENCE TO RELATED APPLICATIONS

This application claims the benefit of U.S. Provisional Application No. 63/176,821, filed Apr. 19, 2021, and U.S. Provisional Application No. 63/232,583, filed Aug. 12, 2021.

### FIELD

The disclosure relates to improved devices and approaches for adjustment or correction of the mandible for treatment and prevention of snoring. The approaches involve gradual titration of the mandible (i.e., lower jaw) from a rearward position to a more forward position to open the oropharyngeal airway and improve breathing.

### BACKGROUND

Snoring and sleep apnea may result from a rearwardly positioned mandible that causes narrowing of the airway and difficulty breathing. Snoring may be associated with loss of sleep, and associated health problems, for the snorer or people nearby. Sleep apnea is a potentially serious sleep disorder in which breathing repeatedly stops and starts. The primary types of sleep apnea include obstructive sleep apnea, central sleep apnea, and complex sleep apnea syndrome. Obstructive sleep apnea occurs when the muscles in the back of the throat relax and the mandible transitions rearward from the maxilla, which causes the airway to narrow or close during inhalation. This causes blood oxygen levels to drop, which may cause the individual to wake and have trouble staying asleep. Central sleep apnea is less common compared to obstructive sleep apnea and occurs when signals delivered from the brain to muscles responsible for breathing are not effectively transmitted. Complex sleep apnea syndrome may be characterized by the presence of both obstructive sleep apnea and central sleep apnea.

A number of treatment approaches require a medical diagnosis of sleep apnea. Because access to healthcare may not be reliable for many individuals, including individuals in developed and under-developed countries, in practice, many people are unable to treat their sleep apnea. In addition, many such approaches are unsatisfactory for various reasons and are often not suitable for managing snoring, which may not necessarily come with a medical diagnosis of sleep apnea. Previous devices, including customizable devices, are overly complex or expensive to produce and may be difficult to use or may increase a risk of injury to the patient due to an increased risk of misconfiguration or misuse. These devices also generally require increased management and oversight by medical personnel, further contributing to cost and lack of access.

For example, U.S. Pat. No. 6,604,527 B1 relates to a mandibular advancement device that includes an upper plate and a lower plate, the upper plate including upper flanges and the lower plate including lower flanges. The leading edge of the upper flanges provide engagement surfaces complementing the engagement surfaces of the lower flanges. The device, which may be for sleep apnea, is impractical and overly expensive to provide to the patient and is also not easily or economically customizable to the individual's mouth, teeth, or jaws.

As another example, U.S. Pat. No. 8,833,374 B2 relates to an intra-oral mandibular advancement appliance that may include a material that may be form-fitting to the teeth after heating and includes a lower tray assembly that may be advanced forwardly independent of an upper tray assembly. The device is overly complex and cannot enter an open configuration, and as such, the subject would not be able to open their mouth during use of the device.

As another example, US 2018/0360646 A1 relates to a mandibular advancement device that includes a lower bite block, lower flanges, an upper bite block, and upper flanges. A shell includes rails for sliding movement of the flanges relative to the bite blocks and includes teeth which are constructed, and which operate, similar to a cable-tie or tie-wrap, such that the flanges ratchet unidirectionally. As the wearer slides the flanges along the rails, the teeth ratchet to change the positions of the flanges in a manner which cannot be reversed without removing the device from the mouth and continuing the sliding ratchet movement until the rails are removed from the shell. The device may become misconfigured before or during use, resulting in the need to remove the device from the mouth and readjust to a different configuration with the ratcheting mechanism. In some instances, the wearer may not necessarily know if the device becomes misconfigured to the point where it needs to be removed and reset, and there may be an increased risk of injury to the patient, for example, if the mandible is forwardly positioned to an extreme degree. This risk and complex operation of the device necessitates extensive oversight of the patient by a health care or dental care professional and prevents the patient from having a degree of self-care in managing their obstructive sleep apnea.

As another example, US 2020/0163795 A1 relates to a fully customized mandibular adjustment device that includes an upper splint with followers and lower splints with contact surfaces, such that the followers and contact surfaces engage each other. The lower splints are exchanged with other lower splints to control advancement of the mandible. The device is fully customized and complex and would be difficult to produce economically. In addition, identification of a lower splint with the correct contact surfaces may be difficult for the user due to the similarity in appearance among different lower splints and contact surfaces. This difficulty may lead to selection of the wrong lower splint, misconfiguration of the device, and misuse of the device and a heightened risk of injury to the patient. Again, there would be a need for extensive oversight of the patient by a health care or dental care professional.

As yet another example, WO 2017/173485 A1 relates to a mandibular repositioning device that includes an upper dental plate with locating tabs and a lower dental plate with receiving formations for positioning the plates relative to each other in an anterior-posterior (AP) position of the mandibular arch relative to the maxillary arch. The device may not be able to reliably hold the mandible in place during use because the device is described as being used in combination with a head collar. The head collar may allow a strained opening of the mouth, however, if the jaw muscles are relaxed, the head collar closes the wearer's mouth to prevent the wearer from easily opening their mouth as may occur during sleeping, drinking, or speaking. In addition, the adjustment mechanism may not be set to the correct setting by the wearer and again, like other existing devices, there would be a need for extensive oversight of the patient by a health care or dental care professional to ensure correct use of the device.

Accordingly, there is a need for improved devices, kits, systems, and methods for forward adjustment of a mandible of an individual for prevention, diagnosis, and treatment of sleep apnea, snoring, and other conditions, diseases, and disorders characterized at least in part by a rearwardly positioned mandible. There is also a need for economically produced mandible advancement devices that are easy to use and improve access to care for snoring and sleep apnea, and which also enable self-care of snoring and sleep apnea to a greater degree compared to previous efforts. The present invention addresses these unmet needs.

### SUMMARY

In various aspects, the disclosure provides improved mandibular advancement devices (MADs) that enable effective titration of the mandible to open and stabilize the oropharyngeal airway. A MAD includes a pair of guards (i.e., an upper guard for engaging upper teeth and a lower guard for engaging lower teeth) and an adjustment piece of a variable length that includes left and right abutment members thereon. The upper and/or lower guard may be easily custom fitted to the individual's teeth (or gums, dentures, implants) to reduce movement of the guards relative to the teeth during use. The adjustment piece is attached to a first guard of the pair of guards and the left and right abutment members engage left and right members of a second guard of the pair of guards to limit rearward movement of the mandible. When the MAD is worn, regardless of whether the device is closed (i.e., closed jaw or mouth) or open (i.e., open jaw or mouth), the left and right members, which may be fin shaped, may contact, and be positioned anterior to, the left and right abutment members. In this manner, the device maintains a forward position of the mandible while also allowing the individual to comfortably speak and drink in the open configuration, and to sleep in the closed configuration. Because the wearer can open and close their jaw freely, there is also less discomfort at the temporomandibular joint (TMJ).

The dimensions of the adjustment piece help to define placement of the abutment members and, in turn, placement of the guard opposite the guard to which the adjustment piece is connected, always to limit rearward movement of the lower guard. For example, if the adjustment piece were to be connected to the lower guard, a length of the adjustment piece would determine placement of members which, in turn, engage members of the upper guard to limit rearward movement of the lower guard. As disclosed herein, the adjustment piece is connected to the upper guard and the abutment members affect placement of left and right members of the lower guard to limit rearward movement of the lower guard; however, alternate configurations are envisioned without departing from the scope of the disclosure.

The adjustment piece may be one of a plurality of adjustment pieces of varying dimensions. The individual, either independently or based on the advice of a professional, may select an appropriate adjustment piece from the plurality of adjustment pieces to incrementally adjust the mandible and gradually open the oropharyngeal airway without overextending the lower jaw. In this manner, breathing during sleep is improved without risking injury or discomfort due to dramatic or improper mandibular advancement.

The adjustment pieces may have particular sizes, shapes, or dimensions to define different configurations of the guards. The different configurations of the guards may be used to incrementally and gradually adjust or titrate the mandible, e.g., as part of a stepwise program. One or both of the guards and/or the adjustment pieces may include identifying indicia thereon, such as coloring, lettering, numbering, and the like to facilitate selection of the appropriate adjustment piece to ensure correct adjustment of the mandible, or to ensure correct maintenance of a position of the mandible, such as may be needed after a completed mandibular titration. In this manner, the individual may confidently use the MAD and benefit from improved breathing during sleep with reduced oversight from a health care provider or a dental care provider.

The adjustment piece being used for a particular adjustment may be applied to the upper guard by inserting an elongated strip of the adjustment piece into a groove of the upper guard, attaching a left connector of the upper guard to a left connector of a left abutment member of the adjustment piece, and attaching a right connector of the upper guard to a right connector of a right abutment member of the adjustment piece. After attachment of the adjustment piece to the upper guard, the left abutment member abuts against a left member of the lower guard and the right abutment member abuts against a right member of the lower guard. These abutment contacts result in a posterior limit for rearward movement of the lower guard, and therefore the mandible, and prevent excessive rearward movement of the mandible to keep the airway open during sleep.

The disclosure also provides kits and systems which may comprise the MAD, and which may also comprise additional components, such as a wand, a plurality of adjustment pieces, instructional materials, etc. The wand may be used to engage a wand attachment point of a guard of the pair of guards for submersion of the MAD (e.g., into boiling water) for custom-fitting the MAD to the individual. In this manner, the MAD may snugly fit to the maxillary teeth and/or the mandibular teeth to minimize discomfort and improve adherence of the MAD to the teeth to hold the mandible in position during use. Accordingly, the disclosure provides all-in-one kits and systems for relief of snoring and/or treatment of sleep apnea. The manufacture, packaging, sale, and delivery of the devices, kits, and systems may be readily scaled to meet demand as needed.

The disclosure also provides methods for adjusting a mandible of an individual, comprising providing a mandibular advancement device (MAD); applying the MAD that includes a first adjustment piece to an oral cavity of an individual for a first adjustment of the mandible; and applying the MAD that includes a second adjustment piece to the oral cavity of the individual for a second adjustment of the mandible. The first adjustment piece differs from the second adjustment piece with respect to a dimension which impacts placement of the left and right abutment members of the adjustment piece and the corresponding posterior limits for the mandible. In this manner, the length of the adjustment piece, which may vary or be variable as part of a titration to adjust placement of the mandible, is a primary factor that determines the degree of mandible advancement.

Another object of the present invention is to provide mandibular advancement devices, kits, systems, and methods which may be readily and economically manufactured using any suitable materials and manufacturing processes.

Other objects, features and advantages of the present invention will become apparent from the following detailed description taken in conjunction with the accompanying drawings.

### BRIEF DESCRIPTION OF THE DRAWINGS

Although the characteristic features of the invention will be particularly pointed out in the claims, the invention itself and manners in which it may be made and used may be better understood after a review of the following description, taken in connection with the accompanying drawings, wherein like numeral annotations are provided throughout.

FIG. 1A shows a front view of an upper guard of an exemplary mandibular advancement device (MAD) of the invention.

FIG. 1B shows a top view of the upper guard.

FIG. 1C shows a left view of the upper guard, without an adjustment piece attached thereto.

FIG. 1D shows a right view of the upper guard, without the adjustment piece attached thereto.

FIG. 2A shows a bottom view of a lower guard of the exemplary MAD.

FIG. 2B shows a front view of the lower guard.

FIG. 2C shows a top view of the lower guard.

FIG. 3A shows a front view of an adjustment piece of the exemplary MAD.

FIG. 3B shows a left view of the adjustment piece.

FIG. 3C shows a top view of the adjustment piece.

FIG. 3D shows a perspective view of a plurality of adjustment pieces, as may be provided, and used, with the MAD.

FIG. 4A shows a perspective view of a wand that may be used with the MAD, as may be provided, and used, with the MAD.

FIG. 4B shows a perspective view of the wand attached to the lower guard of the MAD.

FIG. 5A shows a right view of the MAD closed in a first configuration with a set of artificial teeth.

FIG. 5B shows a right view of the MAD closed in a second configuration with the artificial teeth.

FIG. 5C shows a right view of the MAD closed in a third configuration with the artificial teeth.

FIG. 5D shows a front view of the MAD open in the first configuration with the artificial teeth.

FIG. 5E shows a right view of the MAD open in the first configuration with the artificial teeth.

FIG. 6A shows steps of a method of providing a MAD to an entity for mandibular adjustment.

FIG. 6B shows steps of a method of fitting the MAD to a subject and providing, to the subject, adjustment pieces for a mandibular titration.

FIG. 6C shows steps of a method of titrating the mandible from a first position to more anterior positions with a first adjustment, a second adjustment, and additional adjustment(s).

FIG. 7 shows a diagram of teeth of an individual.

### DETAILED DESCRIPTION

Reference is made herein to the attached drawings. Like reference numerals may be used in the drawings to indicate like or similar elements of the description. The figures are intended for representative purposes, are not drawn to scale, and should not be considered limiting.

### I. Definitions

Unless otherwise defined herein, terms and phrases used in connection with the present disclosure shall have the meanings that are commonly understood by those of ordinary skill in the art.

Where a reference is made to a singular noun, whether with or without use of an indefinite or definite article (e.g., “a”, “an”, or “the”), this includes a plural of that noun unless something else is specifically stated. Furthermore, the terms first, second, third, and the like in the description and in the claims, are used for distinguishing between elements and not necessarily for describing a sequential or chronological order. It is to be understood that the terms so used are interchangeable under appropriate circumstances and that the embodiments of the disclosure described herein are capable of operation in other sequences than described or illustrated herein.

As used herein, the term “about” refers to the usual error range for the respective value readily known to the skilled person in this technical field. Reference to “about” a value or parameter herein includes and describes implementations and embodiments that are directed to that value or parameter per se.

As used herein, terms that may indicate an ability of an element to have a property or characteristic as part of a state of the element include states of the element in which it has the property or characteristic (e.g., is attachable and is attached) as well as states of the element in which it does not have the property or characteristic (e.g., is attachable but is not attached), unless something else is specifically stated. If an element is described as having a property or characteristic (e.g., is attached), this includes elements that may conditionally have the property or characteristic (e.g., may be conditionally attached) as well as elements that may unconditionally have the property or characteristic (e.g., is unconditionally attached), unless something else is specifically stated.

As used herein, the terms “subject”, “patient”, “individual”, “wearer”, and the like, generally refer to a human being having a mandible or lower jaw that may be advanced, titrated, or adjusted with use of a MAD of the present disclosure, and under certain circumstances, these and other similar terms may be interchangeable.

### II. Mandibular Advancement Devices (MADs)

The disclosure provides improved mandibular advancement devices (MADs) that enable effective titration of the mandible to open and stabilize the oropharyngeal airway. The use of an upper guard and a lower guard enable the MAD to adhere to the teeth (or gums, dentures, implants) of the individual during use of the MAD. The upper guard contacts the upper (maxillary) teeth and the lower guard contacts the lower (mandibular) teeth.

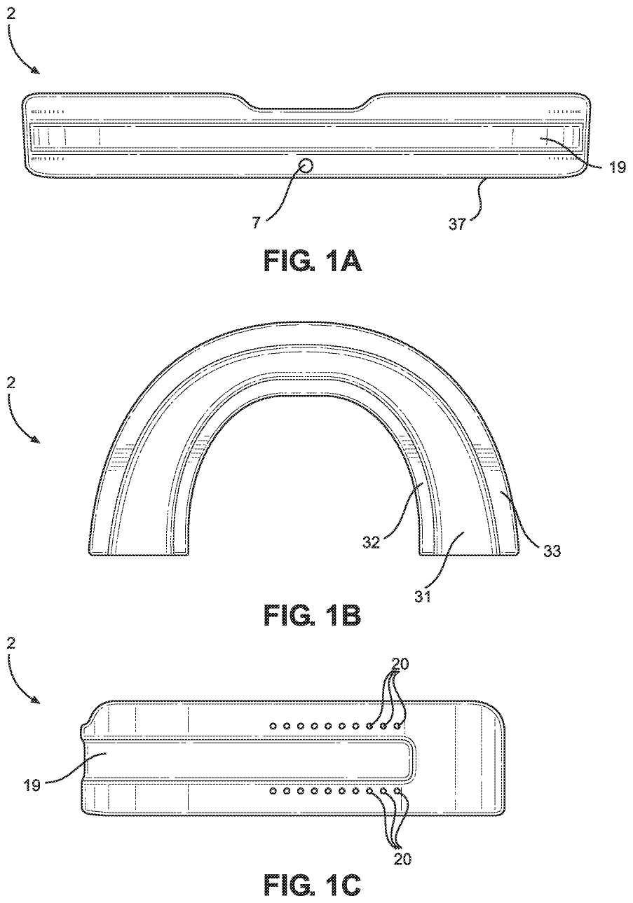

Referring now to FIGS. 1A, 1B, 1C, and 1D, there are depicted several views of an upper guard of an exemplary mandibular advancement device (MAD) of the invention, with a front view (FIG. 1A), a top view (FIG. 1B), a left view without an adjustment piece attached (FIG. 1C), and a right view without the adjustment piece attached (FIG. 1D).

Generally, a MAD comprises an upper guard 2 that is configured to reversibly engage the maxillary teeth of an individual. The upper guard 2 may be comprised of a rigid outer material, such as a firm or hard plastic or polymer, affixed to a temperature moldable inner material of a bite block 31 of the upper guard, such as a thermoplastic or synthetic resin, which softens when heated and hardens when cooled to enable teeth-contacting portions of the upper guard 2 to be custom fitted to the maxillary teeth and contour of the individual's mouth, without a need for the individual to visit a dentist office. In this manner, the upper guard 2 does not fall out of the mouth or become displaced during use.

The upper guard 2 may include a wand attachment point 7 thereon, such as a threaded aperture, configured to receive a wand therein so that the upper guard 2 can be lowered into a hot liquid, such as boiling water, to soften the inner material prior to fitting the upper guard 2 to the maxillary teeth. After the inner material cools, it may retain an impression on the bite block 31 that is characteristic of the maxillary teeth and may thereby more snugly fit within the mouth and enable secure adjustment of the mandible without the upper guard 2 becoming loose or dislodged during use.

The upper guard may substantially or completely cover the maxillary teeth to ensure grip by way of an inner wall 32 and an outer wall 33 which extend upward from the bite block 31 and envelop all or part of the maxillary teeth when applied thereto. During use, a bottom portion 37 of the upper guard 2 may engage a top portion of a lower guard of the MAD.

The upper guard 2 may be comprised of a U-shaped arch configured to fit around the teeth and includes a groove 19 that extends along a surface (e.g., an outer perimeter surface) of the upper guard 2. The groove 19 is configured to reversibly accept an adjustment piece therein such that the adjustment piece is flush or substantially flush with the upper guard 2 when applied thereto, and the upper guard 2 may also include a plurality of left connectors 20 and a plurality of right connectors 21 thereon. It should be appreciated that not all connectors (20, 21) are necessarily indicated with lead lines in the figures; see small circles (20, 21, others) which indicate connectors in the figures.

The plurality of right connectors 21 correspond to the plurality of left connectors 20, and generally, the left and right connectors (20, 21) are configured for securing the adjustment piece to the upper guard 2 and within the groove 19 by connecting the adjustment piece at both the left and right sides of the upper guard 2. In this manner, when a posterior bias of the mandible applies a force to the abutment members of the adjustment piece, the adjustment piece does not slip out of the groove 19 and allow the mandible to move rearwardly, which may cause the airway to close and interrupt breathing during sleep.

In the shown embodiment, the upper guard 2 includes ten left connectors 20 and ten right connectors 21, however, other quantities of connectors may be utilized without departing from the scope of the invention. The connectors (20, 21) may be evenly spaced to provide gradual and regular adjustment of the mandible by using differently sized adjustment pieces over time. In embodiments, each connector (e.g., each connector 20, each connector 21) may be spaced about 1 mm apart from each adjacent connector (e.g., each adjacent connector 20, each adjacent connector 21). The connectors (20, 21) may be comprised of any suitable connection type, but in the shown embodiment, are comprised of individual apertures configured to accept corresponding pins of the adjustment pieces therein. In this manner, the adjustment pieces may be securely and reversibly attached to the upper guard 2 to configure the upper guard 2 for use and may clip or snap in place for secure placement.

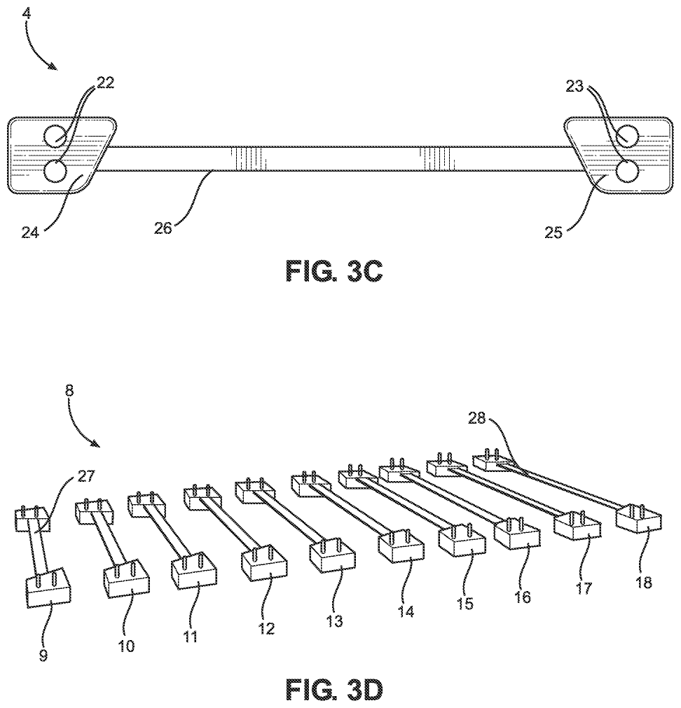

The adjustment piece may be reversibly attached to the groove 19 by inserting an elongated strip of the adjustment piece into the groove 19, attaching the plurality of left connectors 20 (e.g., apertures) of the upper guard 2 to a plurality of left connectors of a left abutment member of the adjustment piece (e.g., pins 22 of FIGS. 3A-3C), and attaching the plurality of right connectors 21 (e.g., apertures) of the upper guard 2 to a plurality of right connectors of a right abutment member of the adjustment piece (e.g., pins 23 of FIGS. 3A-3C). In this manner, the adjustment piece is securely and reversibly applied to the upper guard 2 to reduce or prevent slippage of the adjustment piece therefrom during use. When it is time to use a different-sized adjustment piece, the previous adjustment piece can be removed from the upper guard by removing the left and right connections between the upper guard and the previous adjustment piece and peeling the previous adjustment piece out of the groove 19 to remove it from the upper guard 2.

In embodiments, the upper guard 2 (see FIGS. 1A, 1B, 1C, and 1D) and/or the adjustment piece(s) (see FIGS. 3A, 3B, 3C, and 3D) comprises coloring or indicia thereon. These items may include integer numbering indicia, colors, textures, surface patterns, etc., to clearly indicate each of a plurality, e.g., one to ten (‘1’ to ‘10’) total positions at which the adjustment piece may be attached to connectors (20, 21) of the upper guard 2. The adjustment piece may be one of a plurality of adjustment pieces (e.g., ten total) for use with the upper guard 2, and the different adjustment pieces may vary in length such that they attach to the upper guard 2 at corresponding positions of the upper guard 2. In embodiments in which both the upper guard 2 and the plurality of adjustment pieces includes coloring and/or indicia thereon, the markings may be the same or otherwise correspond between a particular adjustment piece and the corresponding position it is to be attached to the upper guard 2. For example, an adjustment piece for a fourth position may include indicia thereon (e.g., ‘4’) which informs the individual to attach the adjustment piece to the upper guard 2 at the corresponding position (e.g., ‘4’) on the upper guard 2. In this manner, the individual can easily and correctly select the correct adjustment piece to adjust the mandible and little or no oversight from medical or dental professionals is needed.

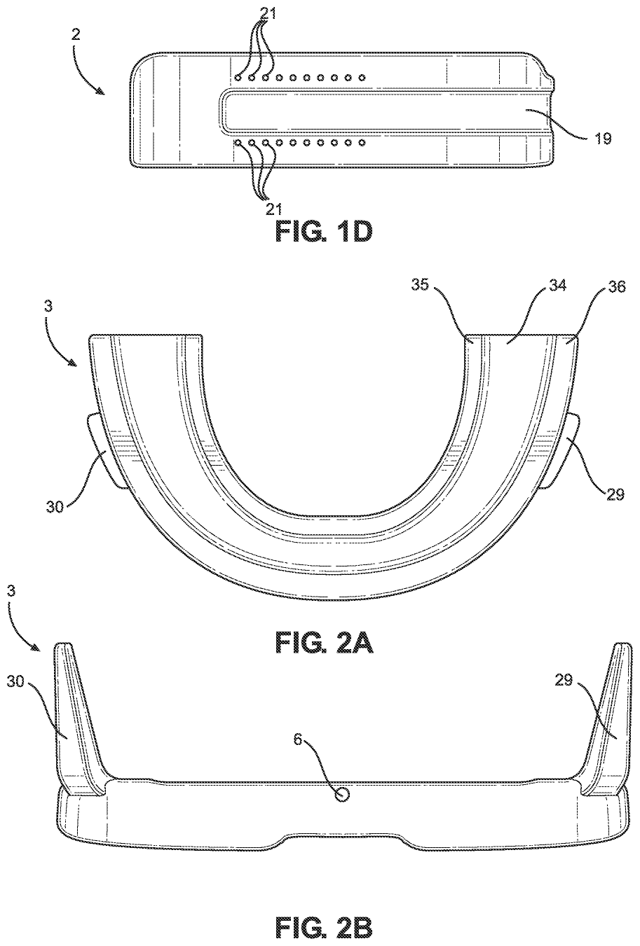

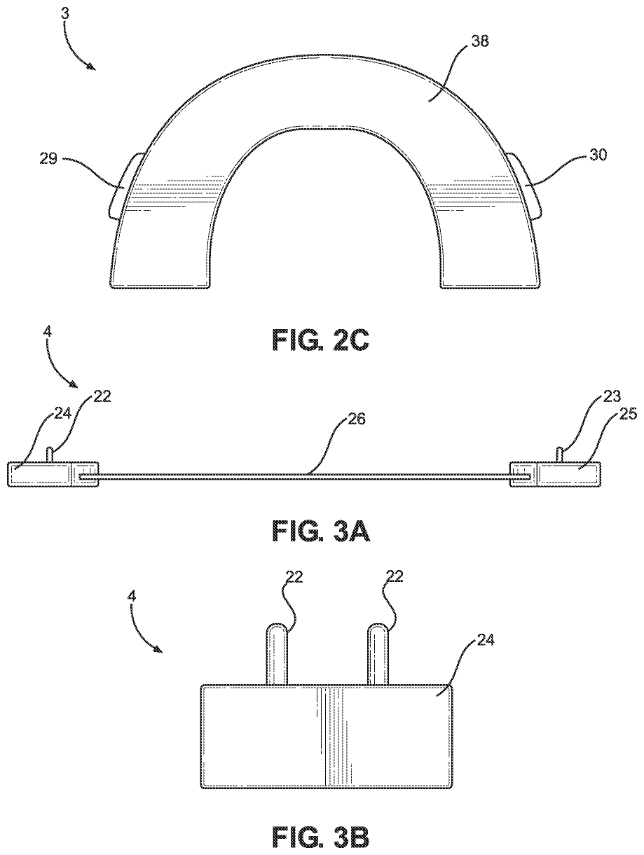

Referring now to FIGS. 2A, 2B, and 2C, there are depicted several views of a lower guard of the exemplary MAD, including a bottom view (FIG. 2A), a front view (FIG. 2B), and a top view (FIG. 2C).

Generally, the MAD comprises a lower guard 3 that is configured to reversibly engage the mandibular teeth of the individual. Like the upper guard, the lower guard 3 may be comprised of a rigid outer material, such as a firm or hard plastic or polymer, affixed to a temperature moldable inner material of a bite block 34 of the lower guard, such as a thermoplastic or synthetic resin, which softens when heated and hardens when cooled to enable teeth-contacting portions of the lower guard 3 to be custom fitted to the mandibular teeth and contour of the individual's mouth, without a need to visit a dentist office. In this manner, the lower guard 3 does not fall out of the mouth or become displaced during use.

The lower guard 3 may include a wand attachment point 6 thereon, such as a threaded aperture, configured to receive the wand therein so that the lower guard 3 can be lowered into a hot liquid, such as boiling water, to soften the inner material prior to fitting the lower guard 3 to the mandibular teeth. After the inner material cools, it may retain an impression on the bite block 34 that is characteristic of the mandibular teeth and may thereby more snugly fit within the mouth and enable secure adjustment of the mandible without the lower guard 3 becoming loose or dislodged during use.

The lower guard may substantially or completely cover the mandibular teeth to ensure grip by way of an inner wall 35 and an outer wall 36 which extend upward from the bite block 34 and envelop all or part of the mandibular teeth when applied thereto. During use, a top portion 38 of the lower guard 3 may engage the bottom portion 37 of the upper guard of the MAD.

Like the upper guard, the lower guard 3 may be comprised of a U-shaped arch configured to fit around the teeth. The lower guard 3 may include a left member 29 at a left portion thereof and a right member 30 at a right portion thereof, which extend upward from the left portion and the right portion, respectively. The members (29, 30) may each be at least approximately triangular in shape or otherwise collectively may resemble a pair of fins, but generally may assume any suitable shape without departing from the scope of the invention.

During use of the MAD, the left abutment member of the adjustment piece abuts against the left member 29, and the right abutment member of the adjustment piece abuts against the right member 30. The members (29, 30) may be statically positioned or rigidly affixed to the lower guard 3 such that when the posterior surfaces of the members (29, 30) engage the anterior surfaces of the abutment members, they do not slip past. The left and right abutment members of the adjustment pieces, however, may be dynamically positioned and may depend on a property or configuration of the adjustment piece being used. By using static or fixed members (29, 30) and changeable adjustment pieces, the positions of the abutment pieces may be changed without impacting stability or effectiveness of the device. In this manner the mandible is prevented from moving rearward past a desired posterior limit to maintain breathing during sleep and may be gradually titrated to gradually open up the airway as part of an investigative procedure, a therapeutic procedure, or a medical procedure.

Referring now to FIGS. 3A, 3B, and 3C, there are depicted several views of an adjustment piece (FIGS. 3A, 3B, and 3C) and a plurality of adjustment pieces (FIG. 3D) as may be provided as part of the MAD or as part of a kit or a system of the invention. An adjustment piece 4 is configured to engage, e.g., reversibly engage, the upper guard and the lower guard to limit rearward movement of a mandible of an individual during use. The adjustment piece 4 may be one of a plurality of adjustment pieces 8 (e.g., ten total) configured for forward titration of the mandible. To titrate the mandible, two or more adjustment pieces (e.g., any combination of 9, 10, 11, 12, 13, 14, 15, 16, 17, and 18) of the plurality 8 may be used. To maintain a position of the mandible irrespective of or after a titration, a single adjustment piece (e.g., 9, 10, 11, 12, 13, 14, 15, 16, 17, or 18) of the plurality 8 may be used.

The adjustment piece 4 may be structurally unitary and may be comprised of a flexible and strong plastic or polymer. To facilitate production of the adjustment pieces 8, the adjustment pieces 8 may be printed in sheets comprising multiple adjustment pieces and then broken off or removed from the sheet prior to use. In embodiments, a sheet comprising five adjustment pieces, numbered ‘1’ through ‘5’, may be produced, and a second sheet comprising five additional adjustment pieces, numbered ‘6’ through ‘10’, may be produced to provide a range of titration to the MAD.

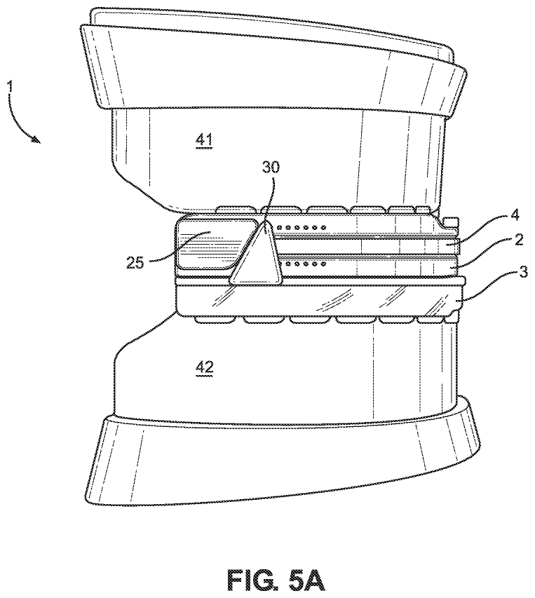

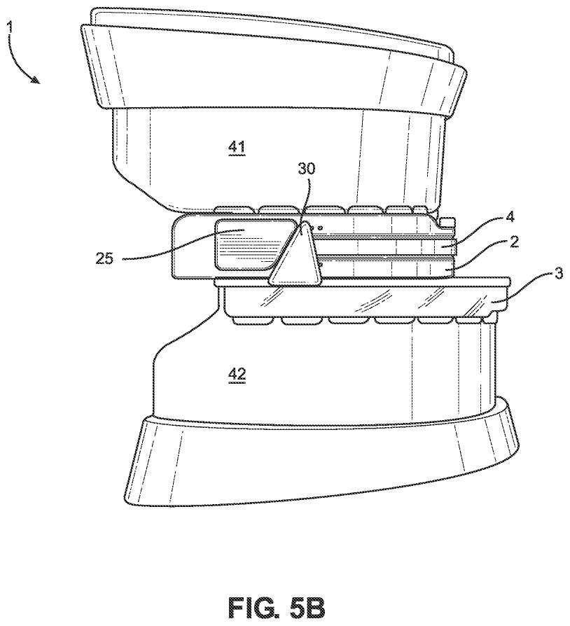

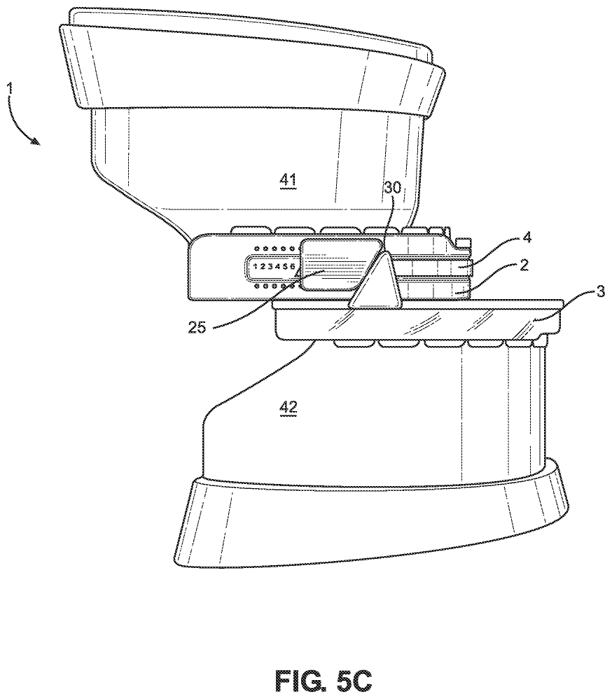

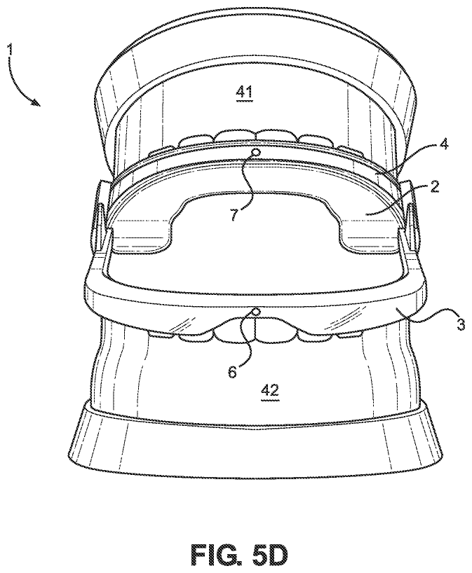

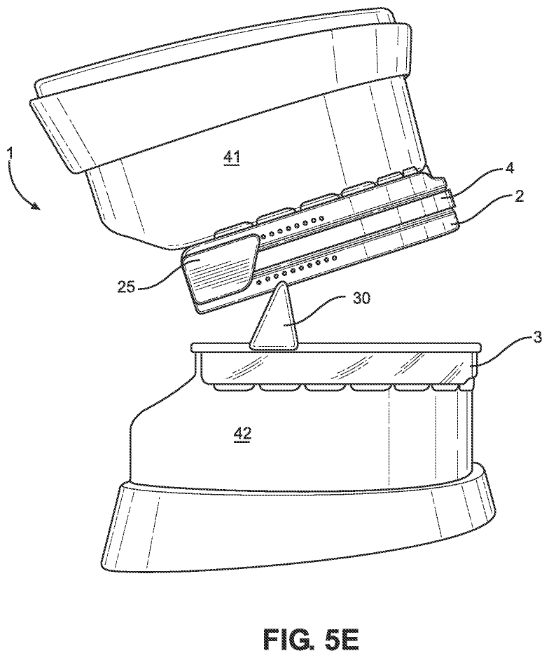

A long adjustment piece 28 may be the longest adjustment piece in the plurality 8 and may be sized and configured to engage the upper guard at position ‘1’ (FIG. 5A) to provide a minimal advancement of the mandible. A short adjustment piece 27 may be the shortest adjustment piece in the plurality 8 and may be sized and configured to engage the upper guard at position ‘10’. Intermediate adjustment pieces (e.g., 10, 11, 12, 13, 14, 15, 16, and 17) may have increasing lengths from the short adjustment piece 27 to the long adjustment piece 28 such that the MAD can be configured for any appropriate advancement of the mandible according to a determination by the individual or a healthcare professional, such as a medical doctor, dentist, sleep specialist, or oral surgeon.

Each adjustment piece 4 (including adjustment pieces 9, 10, 11, 12, 13, 14, 15, 16, 17, and 18 of the plurality 8) includes left and right abutment members (24, 25), which in the shown embodiment may be at least approximately trapezoidal shaped, e.g., shaped as right trapezoids or acute trapezoids, but which generally may assume any suitable shape without departing from the scope of the invention. The abutment members (24, 25) are connected to each other by an elongated strip 26 which may vary in length depending on the adjustment to be performed, e.g., ‘1’ to ‘10’ of ten adjustment pieces (e.g., 9, 10, 11, 12, 13, 14, 15, 16, 17, and 18) and corresponding positions along the groove of the upper guard. In the shown embodiment, each adjustment piece 4 includes the plurality of left connectors (e.g., pins 22) of the left abutment member 24, as well as the plurality of right connectors (e.g., pins 23) of the right abutment member 25 which are configured to be connected with the corresponding connectors of the upper guard to reversibly secure the adjustment piece thereto. In the shown embodiment, the pins (22, 23) are cylindrical, but any suitable shape may be used for pins (22, 23) and corresponding individual apertures of the upper guard without departing from the scope of the invention. Upon insertion of the pins (22, 23) into the corresponding individual apertures of the upper guard, there may be a “click” or “snap” mechanism to provide the individual with tactile or haptic feedback that indicates successful attachment.

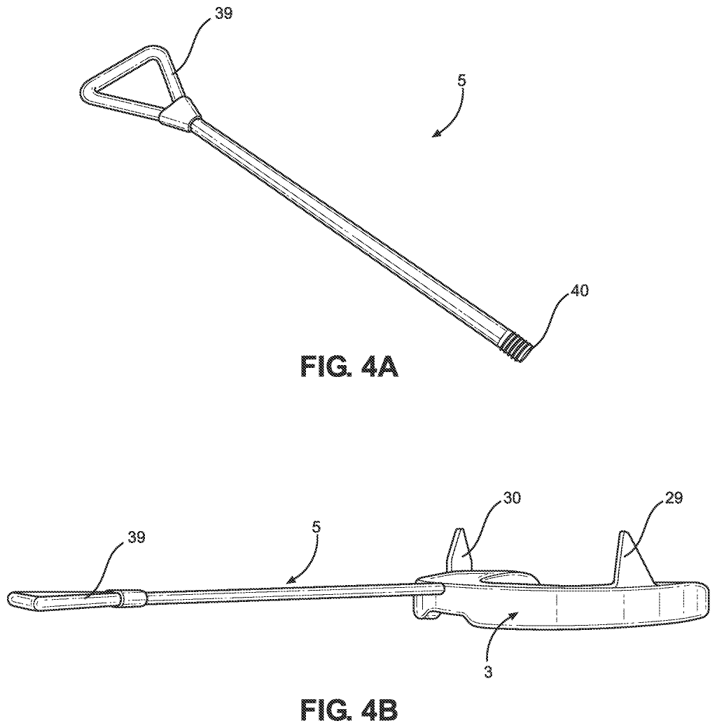

Referring now to FIGS. 4A and 4B, there are depicted a perspective view of a wand that may be used with the exemplary MAD, as may be provided as part of a kit or a system (FIG. 4A), and a perspective view of the wand attached to the lower guard of the exemplary MAD (FIG. 4B). A wand 5 may be comprised of a handle portion 39 (e.g., a proximal portion or a triangular portion) affixed to an attachment point 40 of the wand 5 (e.g., a distal portion or a threaded portion). The wand 5 may be gripped at the handle portion 39, attached to a wand attachment point of the upper guard and/or the lower guard, and used to submerge the upper guard and/or the lower guard into boiling water to soften the temperature moldable inner material for custom fitting to the individual's teeth without risking burning the individual's hands.

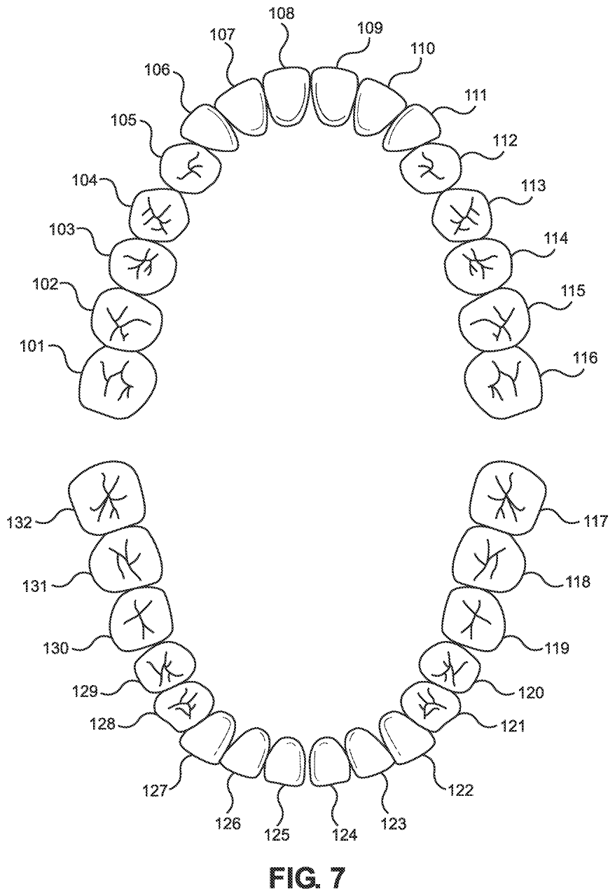

Referring now to FIGS. 5A, 5B, and 5C, there are depicted several right views of the exemplary MAD in use with a set of artificial teeth. While the MAD is shown in use with artificial teeth, it should be appreciated that the exact placement of the MAD may vary with a wearer and between multiple wearers. During use of a MAD 1 of the invention, when the MAD 1 is attached to maxillary teeth 41 and mandibular teeth 42, the abutment members (right abutment member 25 is shown) of the adjustment piece 4 (which is attached to the upper guard 2) abut against the fins (right fin 30 is shown) of the lower guard 3 to prevent rearward movement of the mandible beyond a limit defined by the dimensions of the adjustment piece 4. In addition, in embodiments and during use, the fins (right fin 30 is shown) of the lower guard 3 may be positioned adjacent to, over, or above the first molars of the mandibular teeth for a benefit (see first molars 119 and 130 of lower teeth 117-132 of FIG. 7; upper teeth 101-116 are also shown for clarity). While the right side of the MAD is shown as an example and the left side of the MAD is not explicitly shown in FIGS. 5A-5C, the same or a mirrored configuration applies to the left side of the MAD, i.e., the left abutment member of the adjustment piece 4 abuts against the left fin of the lower guard 3 to prevent rearward movement of the mandible, the same as the right side, as would be understood by the person having ordinary skill in the art. In addition, while not explicitly shown, during use of the MAD by a wearer, the left and right abutment members of the adjustment piece may consistently contact the left and right fins of the lower guard, such that the mandible rearward position is maintained regardless of whether the wearer opens or closes their mouth.

As part of the transition from the first configuration (FIG. 5A) to the second configuration (FIG. 5B), a forward portion of the lower guard 3 is relatively proximal to a forward portion of the upper guard 2 in the first configuration, and upon changing from the first to the second configuration, the forward portion of the lower guard 3 is in front of and relatively distal to the forward portion of the upper guard 2. Similarly, as part of the transition from the second configuration (FIG. 5B) to the third configuration (FIG. 5C), the forward portion of the lower guard 3 is relatively proximal to the forward portion of the upper guard 2 in the second configuration, and upon changing from the second to the third configuration, the forward portion of the lower guard 3 is in front of and relatively distal to the forward portion of the upper guard 2. The relatively forward position of the lower guard 3 in the figures corresponds to the posterior limit for rearward movement of the lower guard, and therefore the mandible, and shows the degree to which the mandible may be stabilized to prevent excessive rearward movement of the mandible to keep the airway open during sleep.

Referring now to FIGS. 5D and 5E, there are depicted front and right views of the exemplary MAD in an open configuration. Generally, the MAD 1 of the invention may enable the individual to open and close their mouth without significant difficulty. Because the upper guard 2 and attached adjustment piece 4 are affixed to the maxillary teeth 41 and the lower guard 3 is affixed to the mandibular teeth 42, the individual can easily open their mouth without needing to remove the MAD 1 beforehand and may easily assume the proper position of the MAD 1 as the mouth is closed again. In this manner, the individual may easily drink, speak, yawn, and perform other activities throughout the night as needed.

The improved mandibular advancement devices (MADs) of the present disclosure enable effective titration of the mandible to open and stabilize the oropharyngeal airway. A MAD includes a pair of guards (i.e., an upper guard for engaging upper teeth and a lower guard for engaging lower teeth) and an adjustment piece which is attached to a guard of the pair of guards and used to limit rearward movement of the mandible of the individual when the MAD is worn by the individual. The MAD is able to be opened to an open configuration such that the individual can maintain near normal physiologic function of the jaw while wearing the device. The MAD is also able to be manufactured at scale and in an economic manner and is ergonomic and configured for improved comfort and a degree of customization. The MAD is also easy to use, owing in part to the clear visual representation of the different sizes of the adjustment pieces and visual indicators to help the individual select the correct adjustment piece for an adjustment step. This, in turn, enables patients to assume a greater role in the care of their snoring and/or sleep apnea and allows them to manage their care with a lesser degree of oversight and intervention by physicians and health care providers. In at least some instances, the MAD may be provided as part of an at-home self-care kit and procedure, with little or no input or direction from medical professionals.

### III. MAD Kits and Systems

Accordingly, in various embodiments, the disclosure also provides kits and systems which may comprise the MAD, and which may also comprise additional components, such as a wand, an initial set of adjustment pieces, replacement adjustment pieces, replacement MAD guards or other components, instructional materials, etc. As described herein, the wand may be used to engage a wand attachment point of a guard of the pair of guards for submersion of the MAD (e.g., into boiling water) for custom-fitting the MAD to the individual. In general, such kits and systems may be provided to doctor offices, dentist offices, pharmacies, stores, medical supply companies, and/or direct to consumers. Accordingly, the disclosure provides all-in-one kits and systems for relief of snoring and/or treatment of sleep apnea that may be provided to any of a variety of different entities based on a given scenario or market need. The manufacture, packaging, sale, and delivery of the devices, kits, and systems may also be readily scaled to meet demand as needed, and due to certain elements of the MAD being common to all MADs, may be relatively lower cost compared to previous efforts in this field.

### IV. MAD Methods

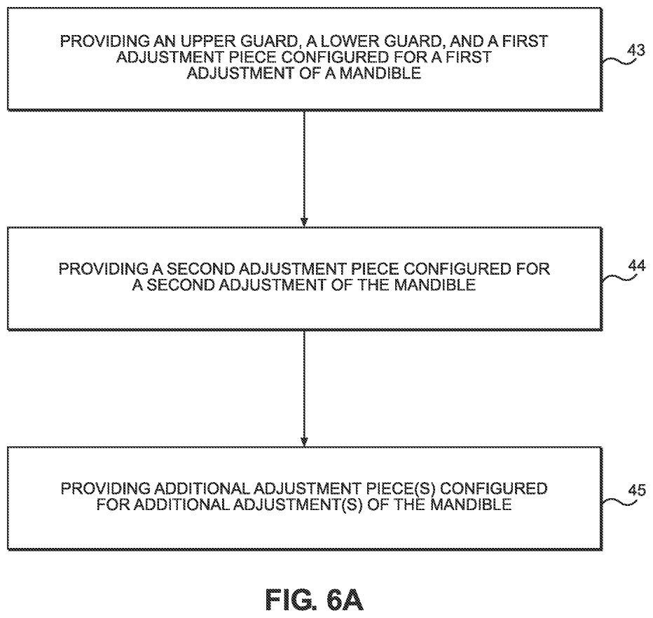

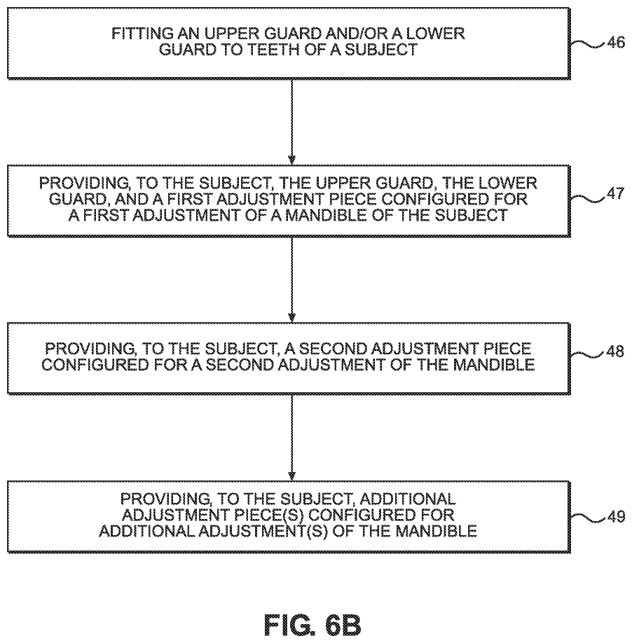

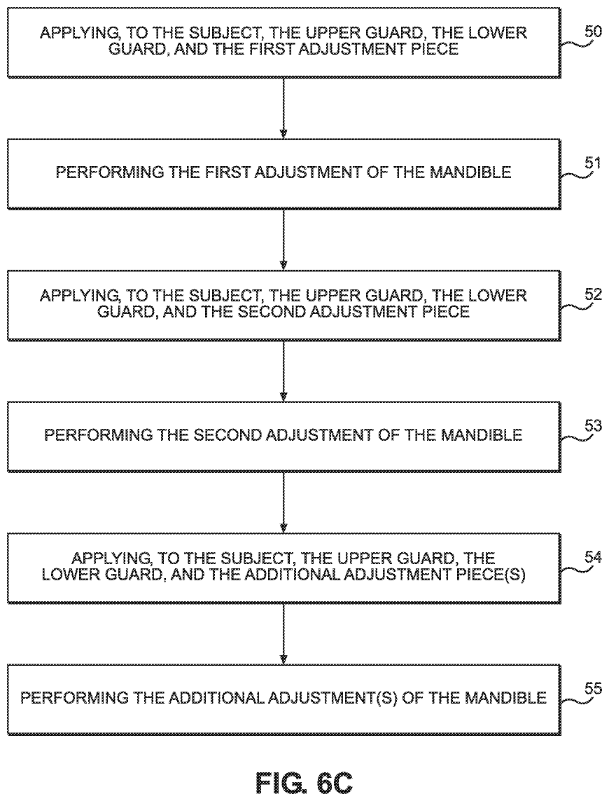

Referring now to FIGS. 6A, 6B, and 6C, there are shown several steps of a method of providing a MAD to an entity for mandibular adjustment (FIG. 6A), fitting the MAD to a subject and providing, to the subject, adjustment pieces for a mandibular titration (FIG. 6B), and titrating the mandible from a first position to more anterior positions with a first adjustment, a second adjustment, and additional adjustment(s) (FIG. 6C). A method for providing a MAD and/or for adjusting a mandible of an individual comprises providing 43, to an entity, a mandibular advancement device (MAD) that includes an upper guard, a lower guard, and a first adjustment piece configured for a first adjustment of a mandible; providing 44, to the entity, a second adjustment piece configured for a second adjustment of the mandible; and in at least some instances, providing 45, to the entity, additional adjustment piece(s) configured for additional adjustment(s) of the mandible.

An individual may have their mandible adjusted by a method that includes the steps shown at FIG. 6B. A method of adjusting or titrating a mandible of a subject may involve fitting 46 an upper guard and/or a lower guard to teeth of the subject; providing 47, to the subject, the upper guard, the lower guard, and a first adjustment piece configured for a first adjustment of the mandible; providing 48, to the subject, a second adjustment piece configured for a second adjustment of the mandible; and, in at least some instances, providing 49, to the subject, additional adjustment piece(s) configured for additional adjustment(s) of the mandible. The method of adjusting or titrating the mandible of the subject may further include steps as shown at FIG. 6C, including applying 50, to the subject, the upper guard, the lower guard, and the first adjustment piece; performing 51 the first adjustment of the mandible; applying 52, to the subject, the upper guard, the lower guard, and the second adjustment piece; performing 53 the second adjustment of the mandible; applying 54, to the subject, the upper guard, the lower guard, and the additional adjustment piece(s); and performing 55, the additional adjustment(s) of the mandible.

The methods of titrating the mandible of the individual are intended to alleviate snoring and/or sleep apnea and generally involve one or more adjustments to the mandible. To affect an adjustment using a first adjustment piece and a second adjustment piece, the first adjustment piece must differ from the second adjustment piece, such as by way of different lengths of elongated strips, as described elsewhere herein. To advance the mandible forward relative to a previous advancement, a length of the first or initial adjustment piece may be greater than a length of the second or subsequent adjustment piece. After the first adjustment piece is used for the first adjustment, it is removed and replaced with the second adjustment piece to be used for the second adjustment. Additional subsequent adjustments may continue to move the mandible forward or, if needed, may move the mandible rearward, for example, if it is determined that the mandible was overly adjusted, and a less significant adjustment may be made without impacting breathing during sleep.

Accordingly, a method of adjusting or titrating the mandible may comprise inserting an elongated strip of a first adjustment piece or a second adjustment piece into a groove that extends along a surface of the upper guard; attaching a left connector of the upper guard to a left connector of a left abutment member of the first adjustment piece or the second adjustment piece; and attaching a right connector of the upper guard to a right connector of a right abutment member of the first adjustment piece or the second adjustment piece. When the MAD is in a first configuration that includes the first adjustment piece, the left abutment member of the first adjustment piece abuts against a left fin of a left portion of the lower guard and the right abutment member of the first adjustment piece abuts against a right fin of a right portion of the lower guard, and when the MAD is in a second configuration that includes the second adjustment piece, the left abutment member of the second adjustment piece abuts against a left fin of a left portion of the lower guard and the right abutment member of the second adjustment piece abuts against a right fin of a right portion of the lower guard.

Generally, in such a method, the first configuration differs from the second configuration with respect to a position of the lower guard relative to the upper guard. In this manner, the titration may be effectively performed. When the MAD is in the first configuration a forward portion of the lower guard may be relatively proximal to a forward portion of the upper guard, and when the MAD is in the second configuration the forward portion of the lower guard may be in front of and relatively distal to the forward portion of the upper guard.

The foregoing descriptions of specific embodiments of the present invention have been presented for purposes of illustration and description. They are not intended to be exhaustive or to limit the present invention to the precise forms disclosed, and modifications and variations are possible in view of the above teaching. The exemplary embodiment was chosen and described to best explain the principles of the present invention and its practical application, to thereby enable others skilled in the art to best utilize the present invention and its embodiments with modifications as suited to the use contemplated.

It is therefore submitted that the present invention has been shown and described in the most practical and exemplary embodiments. It should be recognized that departures may be made which fall within the scope of the invention. With respect to the description provided herein, it is submitted that the optimal features of the invention include variations in size, materials, shape, form, function and manner of operation, assembly, and use. All structures, functions, and relationships equivalent or essentially equivalent to those disclosed are intended to be encompassed by the present invention.

Any discussion of documents or subject matter included in the present disclosure is not to be taken as an admission that any or all of these materials form part of the prior art base or were common general knowledge in the field relevant to the present disclosure as it existed before the priority date of each claim of this and any continuing application.

I claim:

1) A mandibular advancement device (MAD), comprising:
a pair of guards that includes an upper guard and a lower guard; and
an adjustment piece that includes left and right abutment members;
wherein when worn by an individual, the left and right abutment members of the adjustment piece engage a guard of the pair of guards to limit rearward movement of the mandible of the individual.

2) The MAD of claim 1, wherein the upper guard and/or the lower guard includes a temperature moldable material on a bite block thereof for a custom fit of the guard(s) to the teeth; and wherein the upper guard and/or the adjustment piece comprises coloring and/or indicia thereon to help the individual identify an adjustment piece out of a plurality of adjustment pieces.

3) The MAD of claim 1, wherein the upper guard comprises a groove that extends along a surface of the upper guard and reversibly accepts the adjustment piece therein to help secure the adjustment piece in place.

4) The MAD of claim 1, wherein the upper guard comprises a left connector at a left portion thereof and a right connector at a right portion thereof;
wherein the left connector of the upper guard is configured to connect with a left connector of the adjustment piece; and
wherein the right connector of the upper guard is configured to connect with a right connector of the adjustment piece.

5) The MAD of claim 1, wherein the adjustment piece is attachable to the upper guard to limit rearward movement of the mandible as part of a forward adjustment of the mandible.

6) The MAD of claim 5, wherein multiple adjustment pieces of different lengths define a plurality of positions of the lower guard relative to the upper guard and a corresponding plurality of positions of the mandible for the forward adjustment of the mandible.

7) The MAD of claim 1, wherein the left abutment member and the right abutment member are connected via an elongated strip.

8) The MAD of claim 7, wherein a length of the elongated strip corresponds to a position of the lower guard relative to the upper guard and a corresponding position of the mandible when worn.

9) The MAD of claim 7, wherein the left abutment member abuts against a left fin of a left portion of the lower guard and the right abutment member abuts against a right fin of a right portion of the lower guard.

10) The MAD of claim 7, wherein the upper guard comprises a left connector at a left portion thereof and a right connector at a right portion thereof;
wherein the left connector of the upper guard is configured to connect with a left connector of the left abutment member; and
wherein the right connector of the upper guard is configured to connect with a right connector of the right abutment member.

11) A mandibular advancement device (MAD), comprising:
an upper guard that comprises a groove that extends along a surface thereof, a left connector at a left portion thereof, and a right connector at a right portion thereof;
a lower guard that comprises a left member at a left portion thereof and a right member at a right portion thereof; and
an adjustment piece that comprises a left abutment member connected with a right abutment member via an elongated strip;
wherein a left connector of the left abutment member connects with the left connector of the upper guard, a right connector of the right abutment member connects with the right connector of the upper guard, and the elongated strip engages with the groove of the upper guard; and
wherein the adjustment piece may be connected with the upper guard such that the left and right members of the lower guard engage the left and right abutment members of the adjustment piece to limit rearward movement of the lower guard relative to the upper guard.

12) The MAD of claim 11, wherein the groove extends along an outer perimeter surface of the upper guard and the left and right connectors of the upper guard are positioned proximal to left and right rear portions of the outer perimeter surface, such that the adjustment piece is positioned along the outer perimeter surface when connected with the upper guard.

13) The MAD of claim 11, wherein the left and right members of the lower guard are each at least approximately triangular in shape or otherwise collectively resemble a pair of fins, and wherein the left and right abutment members of the adjustment piece are each at least approximately trapezoidal shaped.

14) The MAD of claim 11, wherein the left and right members of the lower guard are statically positioned relative to the lower guard, and wherein the left and right abutment members of the adjustment piece are dynamically positioned relative to the upper guard as a result of a dimension of the elongated strip of the adjustment piece.

15) The MAD of claim 14, wherein the dimension of the elongated strip is a variable length that is particular to the adjustment piece.

16) The MAD of claim 14, wherein the adjustment piece is one of a plurality of adjustment pieces of varying lengths that are configured for defining positions of the lower guard relative to the upper guard for titration of a mandible of an individual.

17) A method, comprising:
providing, to an entity, an upper guard, a lower guard, and a first adjustment piece; and
providing, to the entity, a second adjustment piece;
wherein a length of the first adjustment piece is greater than a length of the second adjustment piece;
wherein a first configuration of a mandibular advancement device (MAD) that comprises the upper guard, the lower guard, and the first adjustment piece differs from a second configuration of the MAD that comprises the upper guard, the lower guard, and the second adjustment piece.

18) The method of claim 17, wherein the entity is a health care provider, a dental care provider, an individual, a patient, or a subject.

19) The method of claim 17, further comprising applying the first adjustment piece to the upper guard to produce the first configuration and applying the second adjustment piece to the upper guard to produce the second configuration, respectively, by a plurality of steps, the plurality of steps comprising:
inserting an elongated strip of the first adjustment piece or the second adjustment piece into a groove that extends along a surface of the upper guard;
attaching a left connector of the upper guard to a left connector of a left abutment member of the first adjustment piece or the second adjustment piece; and
attaching a right connector of the upper guard to a right connector of a right abutment member of the first adjustment piece or the second adjustment piece;
wherein the MAD is in the first configuration, the left abutment member of the first adjustment piece abuts against a left fin of a left portion of the lower guard and the right abutment member of the first adjustment piece abuts against a right fin of a right portion of the lower guard;
wherein the MAD is in the second configuration, the left abutment member of the second adjustment piece abuts against a left fin of a left portion of the lower guard and the right abutment member of the second adjustment piece abuts against a right fin of a right portion of the lower guard; and
wherein the first configuration differs from the second configuration with respect to a position of the lower guard relative to the upper guard.

20) The method of claim 19, wherein the MAD is in the first configuration a forward portion of the lower guard is relatively proximal to a forward portion of the upper guard, and wherein the MAD is in the second configuration the forward portion of the lower guard is in front of and relatively distal to the forward portion of the upper guard.
